(19) United States
(12) Patent Application Publication
(10) Pub. No.: US 2024/0279731 A1
(43) Pub. Date: Aug. 22, 2024

(54) MULTI COLOR WHOLE-GENOME MAPPING AND SEQUENCING IN NANOCHANNEL FOR GENETIC ANALYSIS

(71) Applicant: Drexel University, Philadelphia, PA (US)

(72) Inventors: Ming Xiao, Huntingdon Valley, PA (US); Lahari Uppuluri, Philadelphia, PA (US)

(21) Appl. No.: 18/569,789

(22) PCT Filed: Jun. 17, 2022
(86) PCT No.: PCT/US22/34023

§ 371 Date: Dec. 13, 2023

Related U.S. Application Data
(60) Provisional application No. 63/212,357, filed on Jun. 18, 2021.

(51) Int. Cl. C12Q 1/6869; C12N 9/22; C12N 15/11; C12Q 1/6806; G01N 33/58

(52) U.S. Cl. CPC C12Q 1/6869; C12N 9/22; C12N 15/11; C12Q 1/6806; G01N 33/582; C12N 2310/20

(57) ABSTRACT

In one aspect, the invention provides universal multi-color mapping strategy in nanochannels combining conventional sequence-motif labeling system with Cas9 mediated target-specific labeling of any 20-base sequences (20mers) to create custom labels and detect new features. The sequence-motifs are labeled with green fluorophores and the 20mers are labeled with red fluorophores. Using this strategy, it is not only possible to detect the (structural variants) SVs but it is also possible to utilize custom labels to interrogate the features not accessible to motif-labeling, locate breakpoints and precisely estimate copy numbers of genomic repeats. In another aspect, the invention provides CRISPR-Cas9 enabled whole-genome sequencing.

### CROSS-REFERENCE TO RELATED APPLICATIONS

This application claims priority under 35 U.S.C. § 119(e) to U.S. Provisional Patent Application No. 63/212,357, filed Jun. 18, 2021, the disclosures of which is incorporated herein by reference in its entirety.

### SEQUENCE LISTING

The ASCII text file named “046528-7115WO1_Sequence Listing ST25” created on Jun. 17, 2022, comprising 3 Kbytes, is hereby incorporated by reference in its entirety.

### BACKGROUND OF THE INVENTION

Analysis of structural variants (SVs) is important to understand mutations underlying genetic disorders and pathogenic conditions. However, characterizing SVs using short-read, high throughput sequencing technology is difficult. While long-read sequencing technologies are being increasingly employed in characterizing SVs, their low throughput and their high costs discourage widespread adoption. Sequence-motif-based optical mapping in nanochannel is useful in whole-genome mapping and SV detection, but it is not possible to precisely locate breakpoints or estimate copy numbers. Thus, there is an unmet need in the art to develop better genome mapping methods. In one aspect, the present invention addresses this unmet need.

### SUMMARY OF THE INVENTION

In one aspect, the invention is method of mapping a whole genome, wherein the method comprises: a) labeling at least one DNA having a backbone with a first fluorophore by contacting the at least one DNA with a solution comprising the first fluorophore and a labeling enzyme: b) nicking the at least one DNA labeled with the first fluorophore by contacting it with a solution comprising a nickase and at least one single guide RNA (sgRNA) or at least one crisprRNA(crRNA): c) incorporating fluorescent nucleotide(s) at the nicked site(s) of the at least one DNA by contacting it with a solution comprising a DNA polymerase and a mix of nucleotides comprising at least one nucleotide tagged with the second fluorophore: d) staining the backbone of the at least one nicked-labeled DNA of step c) with a DNA backbone stain: e) imaging the at least one DNA of step d) by sequentially exciting the first fluorophore, the second fluorophore, and the DNA backbone stain; and f) analyzing the imaging data to identify the location of the first fluorophore and the second fluorophore for whole genome mapping.

In certain embodiments, the at least one DNA is a genomic DNA (gDNA).

In certain embodiments, the first fluorophore is a green fluorophore.

In certain embodiments, the first fluorophore labels CTTAAG motif(s) of the at least one gDNA.

In certain embodiments, the second fluorophore is a red fluorophore.

In certain embodiments, first fluorophore is exited prior to exiting the second fluorophore. In certain embodiments, the second fluorophore is excited prior to exciting the first fluorophore.

In certain embodiments, the at least one sgRNA or crRNA comprises an about 20 nucleotides long target-recognition sequence.

In certain embodiments, the nickase is Cas9D10A.

In certain embodiments, the backbone is stained with YOYO-1 stain.

In certain embodiments, the method is useful for applications including detecting breakpoints, characterizing repetitive sequence, investigating mutagenesis, and quantifying copy numbers.

In another aspect, the invention provides a method of whole genome sequencing, wherein the method comprises: a) linearizing at least one DNA on a micropatterned surface: b) nicking the at least one DNA by contacting it with a first solution comprising at least one CRISPR-Cas9 nickase/guide RNA (gRNA) complex: c) incorporating fluorescent nucleotide(s) at the nicked site(s) of the at least one DNA of step b) by contacting it with a second solution comprising a DNA polymerase and a mix of nucleotides comprising at least one fluorescently tagged nucleotide: d) imaging the at least one DNA of step c); and e) repeating steps b)-d) with different CRISPR-Cas9 nickase/gRNA complex(es) than that used in previous steps for whole genome sequencing.

In certain embodiments, the first solution comprises up to four different CRISPR-Cas9 nickase/gRNA complexes. In certain embodiments, different colored fluorescent nucleotides are incorporated for different CRISPR-Cas9 nickase/gRNA complexes.

In yet another aspect, the invention comprises a method of whole genome sequencing, wherein the method comprises: a) linearizing at least one DNA on a micropatterned surface: b) labeling the at least one DNA by contacting it with a solution comprising at least one dCas9/gRNA complex tagged with a fluorophore; and c) imaging and sequencing the labeled DNA.

In certain embodiments, the dCas9 present in the dCas9/gRNA complex is tagged with a fluorophore. In certain embodiments, the gRNA present in the dCas9/gRNA complex is tagged with a fluorophore. In certain embodiments, different colored fluorophores are used for tagging dCas9/gRNA complex(es) comprising different gRNAs.

In yet another aspect, the invention provides a method of whole genome sequencing, wherein the method comprises: a) linearizing at least one DNA on a micropatterned surface: b) generating sequencing initiation site(s) (3″-OH ends) along the at least one DNA by contacting it with a first solution comprising at least one Cas9/gRNA complex: c) labeling the at least one DNA from step b) by contacting it with a second solution comprising a DNA polymerase and a mix of fluorophore-tagged reversible terminators: d) imaging the labeled DNA to read signal from the fluorophore: e) reversing the 3′ modification to —OH; f) repeating steps c)-e) and again step c); and) imaging the at least one DNA for whole genome sequencing. In certain embodiments, the at least one DNA is a megabase-long DNA.

In certain embodiments, each reversible terminator comprising different nucleotides are tagged with different fluorophores.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1A shows de novo assembled optical maps of DLE-Cas9 labeled D4Z4 array on Chromosome 4q in NA12878. On the top, 4qA haplotype is seen and, on the bottom, 4qB haplotype can be seen. The wide bar at the top denotes the hg38 reference. The wide bar below the reference represents consensus contigs from the de novo assembly. Individual molecules are represented by the thin lines arranged under the consensus contigs. Vertical ticks on the single molecules indicate labeled DLE sites, while the vertical ticks in the subtelomeric region indicate D4Z4 target-specific red labels. The figures show only a part of all labeled molecules aligned to 4qA and 4qB.

FIG. 1B shows a graph of distances between the red labels plotted against their frequency. Here, the X-axis indicated the distances between the two closest red labels which occurred along the length of the D4Z4 array of a molecule, and the Y-axis indicates the frequency of the recorded distances across all mapped molecules.

FIG. 2A shows de novo assembled optical maps of DLE-Cas9 labeled telomeric repeats array on Chromosome 14q (top panel) and 20q (bottom panel) in NA12878. The wide bar at the top denotes the hg38 reference. The wide bar below the reference represents consensus contigs from the de novo assembly. Individual molecules are represented by the thin yellow lines arranged under the consensus contigs. Vertical ticks on the single molecules (lines) indicate labeled DLE sites, while the vertical ticks at the ends of single molecules indicate telomere red labels. Only a part of all aligned single molecules (lines) are shown in the maps. FIG. 2B shows a plot with measured intensities of red labels at telomere-termini containing single molecules from 14q and 20q arms. Each filled circle represents the total red label intensity of a single molecule. The horizontal bar represents the average measured intensity.

FIGS. 3A-3B LINE-1 insertions detected in a Chr4 haplotype using our DLE-Cas9 approach. Both DLE and red labels are stretch matched in the FIG. 3A shows a haplotype with the 6 kbp line 1 insertion. FIG. 3B shows the second haplotype with no insertion at the same genomic region.

FIGS. 4A-4B are related to CRISPR-Cas9 enabled whole-genome sequencing. FIG. 4A shows the 4-color sequencing scheme. FIG. 4B shows two-color mapping/sequencing on micropatterned surface. gRNA1 TGTAATCCCAGCACTTTGGG (SEQ ID NO: 18) and gRNA2 CGAGACCAGCCTGGCCAACA (SEQ ID NO: 19) are combined in a single cycle. The dots indicate the presence of gRNA1 TGTAATCCCAGCACTTTGGG (SEQ ID NO: 18) and gRNA2 CGAGACCAGCCTGGCCAACA (SEQ ID NO: 19) on single DNA molecules (vertical lines).

FIGS. 5A-5C are related to CRISPR-Cas9 enabled whole-genome sequencing. FIG. 5A shows a schematic of a microdevice containing micropatterned surface for DNA linearization. FIG. 5B shows a base-by-base sequencing strategy based on Cas9/gRNA chemistry. FIG. 5C shows a two-color base-by-base sequencing reaction show reading two bases.

FIGS. 6A-6B are related to quantifying on-off-target labeling efficiency. FIG. 6A show individual DNA molecules (lines with dots showing the green label by DLE and red label by Cas9-gRNA) are assembled into consensus contig (lower bar). The consensus contig is aligned to reference map (upper bar). FIG. 6B is the histogram of red labels of all molecules: the peak indicates the consensus red label locations of all labels at a particular location.

FIG. 7 shows a schematic of DLE-Cas9 multicolor labeling.

### DETAILED DESCRIPTION OF THE INVENTION

The present invention is related to enzymatic labeling strategy for multi-color whole-genome mapping by combining Direct Label Enzyme (DLE-1, Bionano Genomics) with Cas9 mediated nick-labeling reaction. Using this universal strategy, it is possible to target and fluorescently label any 20mers, or the combination of multiple 20 bases across the whole genome, especially in repetitive regions lacking DLE motifs. Custom maps can be generated to enable precise detection of breakpoints and interrogate the repetitive sequences: this enables more in-depth analysis of structural variations than was previously possible.

In order to validate the labeling strategy for multi-color geneome mapping, experiments for quantifying the number of D4Z4 repeats in chromosome 4q, detecting Long non-interspersed Elements 1 (LINE-1) insertions, and estimating the telomere length were performed. D4Z4 is a 3.3 kbp repeat sequence associated with Facioscapulohumeral muscular dystrophy (FSHD). The repeats occur on 4q35 and 10q26 loci lacking certain motifs targeted by DLE enzyme and Nickase (Nt.BspQI) for conventional mapping. Similarly, telomeres in humans are chromosome capping (TTAGGG)n repeats with varying lengths up to 20 kbp. They occur in genomic regions also lacking labeling motifs. LINE-1 insertions are transposable elements and are frequently inserted across the genome. Optical mapping with DLE alone does not differentiate LINE-1s from other insertions. With the DLE-Cas9 methodology shown herein, specific sequences were fluorescently tagged to differentiate LINE-1 insertions from others, the copy numbers of D4Z4 repeats were quantified and the telomere length was estimated.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more in preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

The term, “polynucleotide” includes cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semisynthetic nucleotide bases. Also, included within the scope of the invention are alterations of a wild type or synthetic gene, including but not limited to deletion, insertion, substitution of one or more nucleotides, or fusion to other polynucleotide sequences.

Conventional notation is used herein to describe polynucleotide sequences: the left-hand end of a single-stranded polynucleotide sequence is the 5′-end: the left-hand direction of a double-stranded polynucleotide sequence is referred to as the 5′-direction.

The term “oligonucleotide” or “oligos” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” or “protein” are used interchangeably, and refer to a compound comprised of amino acid residues covalently linked by peptide bonds. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs and fusion proteins, among others. The polypeptides include natural peptides, recombinant peptides, synthetic peptides or a combination thereof. A peptide that is not cyclic will have a N-terminal and a C-terminal. The N-terminal will have an amino group, which may be free (i.e., as a NH2 group) or appropriately protected (for example, with a BOC or a Fmoc group). The C-terminal will have a carboxylic group, which may be free (i.e., as a COOH group) or appropriately protected (for example, as a benzyl or a methyl ester). A cyclic peptide does not have free N- or C-terminal, since they are covalently bonded through an amide bond to form the cyclic structure. Amino acids may be represented by their full names (for example, leucine), 3-letter abbreviations (for example, Leu) and 1-letter abbreviations (for example, L). The structure of amino acids and their abbreviations may be found in the chemical literature, such as in Stryer, “Biochemistry”, 3rd Ed., W. H. Freeman and Co., New York, 1988. tLeu represents tert-leucine. neo-Trp represents 2-amino-3-(1H-indol-4-y)-propanoic acid. DAB is 2,4-diaminobutyric acid. Orn is ornithine. N-Me-Arg or N-methyl-Arg is 5-guanidino-2-(methylamino) pentanoic acid.

“Sample” or “biological sample” as used herein means a biological material from a subject, including but is not limited to organ, tissue, cell, exosome, blood, plasma, saliva, urine and other body fluid, A sample can be any source of material obtained from a subject.

The terms “subject”, “patient”, “individual”, and the like are used interchangeably herein, and refer to any animal, or cells thereof whether in vitro or in situ, amenable to the methods described herein. In certain non-limiting embodiments, the patient, subject or individual is a human. Non-human mammals include, for example, livestock and pets, such as ovine, bovine, porcine, canine, feline and murine mammals. Preferably, the subject is human. The term “subject” does not denote a particular age or sex.

The term “measuring” according to the present invention relates to determining the amount or concentration, preferably semi-quantitatively or quantitatively. Measuring can be done directly.

As used herein the term “amount” refers to the abundance or quantity of a constituent in a mixture.

The term “concentration” refers to the abundance of a constituent divided by the total volume of a mixture. The term concentration can be applied to any kind of chemical mixture, but most frequently it refers to solutes and solvents in solutions.

As used herein, the terms “reference”, or “threshold” are used interchangeably, and refer to a value that is used as a constant and unchanging standard of comparison.

As used herein, “paired-end sequencing” is a sequencing method that is based on high throughput sequencing in which both ends of a DNA fragment are sequenced. Any high throughput DNA sequencing platform may be used, such as those based on the platforms currently sold by Illumina, Oxford Nanopore, Pacific Biosciences, and Roche. Oxford Nanopore's MinION sequencer can generate short to ultra-long (>2 Mb) reads. Illumina has released a hardware module (the PE Module) which can be installed in an existing sequencer as an upgrade, which allows sequencing of both ends of the template, thereby generating paired end reads. Paired end sequencing may also be conducted using Solexa, Oxford Nanopore, or PacBio single-molecule real-time (SMRT) circular consensus sequencing (CCS) technology in the methods according to the current invention. Examples of paired end sequencing are described for instance in US20060292611 and in publications from Roche (454 sequencing).

As used herein the term “sequencing” refers to determining the order of nucleotides (base sequences) in a nucleic acid sample, e.g. DNA or RNA. Many techniques are available such as Sanger sequencing and high-throughput sequencing technologies (also known as next-generation sequencing technologies) such as pyrosequencing based on the “sequencing by synthesis” principle, in which the sequencing is performed by detecting the nucleotide incorporated by a DNA polymerase. Pyrosequencing generally relies on light detection based on a chain reaction when pyrophosphate is released.

A “restriction endonuclease” or “restriction enzyme” refers to an enzyme that recognizes a specific nucleotide sequence (target site) in a double-stranded DNA molecule, and will cleave both strands of the DNA molecule at or near every target site, leaving a blunt or a staggered end.

A “Type-IIs” restriction endonuclease refers to an endonuclease that has a recognition sequence that is distant from the restriction site. In other words, Type IIs restriction endonucleases cleave outside of the recognition sequence to one side. Examples thereof are NmeAlll (GCCGAG(21/19)) and FokI, AlwI, Mme I. Also included in this definition are Type IIs enzymes that cut outside the recognition sequence at both sides.

A “Type IIb” restriction endonuclease cleaves DNA at both sides of the recognition sequence.

“Restriction fragments” or “DNA fragments” refer to DNA molecules produced by digestion of DNA with a restriction endonuclease are referred to as restriction fragments. Any given genome (or nucleic acid, regardless of its origin) can be digested by a particular restriction endonuclease into a discrete set of restriction fragments. The DNA fragments that result from restriction endonuclease cleavage can be further used in a variety of techniques and can, for instance, be detected by gel electrophoresis or sequencing. Restriction fragments can be blunt ended or have an overhang. The overhang can be removed using a technique described as polishing. The term ‘internal sequence’ of a restriction fragment is typically used to indicate that the origin of the part of the restriction fragment resides in the sample genome, i.e. does not form part of an adapter. The internal sequence is directly derived from the sample genome, its sequence is hence part of the sequence of the genome under investigation.

As used herein, “Ligation” refers to the enzymatic reaction catalyzed by a ligase enzyme in which two double-stranded DNA molecules are covalently joined together. In general, both DNA strands are covalently joined together, but it is also possible to prevent the ligation of one of the two strands through chemical or enzymatic modification of one of the ends of the strands. In that case, the covalent joining will occur in only one of the two DNA strands.

“Adapters” or “adaptors” are short double-stranded DNA molecules with a limited number of base pairs, e.g. about 10 to about 30 base pairs in length, which are designed such that they can be ligated to the ends of DNA fragments, such as the linked-paired-end DNA fragments generated by the methods described herein. Adapters are generally composed of two synthetic oligonucleotides that have nucleotide sequences which are partially complementary to each other. When mixing the two synthetic oligonucleotides in solution under appropriate conditions, they will anneal to each other forming a double-stranded structure. After annealing, one end of the adapter molecule is designed such that it is compatible with the end of a DNA fragment and can be ligated thereto: the other end of the adapter can be designed so that it cannot be ligated, but this need not be the case (double ligated adapters). Adapters can contain other functional features such as identifiers, recognition sequences for restriction enzymes, primer binding sections etc. When containing other functional features the length of the adapters may increase, but by combining functional features this may be controlled.

“Adapter-ligated DNA fragments” refer to DNA fragments that have been capped by adapters on one or both ends.

As used herein, “barcode” or “tag” refer to a short sequence that can be added or inserted to an adapter or a primer or included in its sequence or otherwise used as label to provide a unique barcode (aka barcode or index). Such a sequence barcode (tag) can be a unique base sequence of varying but defined length, typically from 4-16 bp used for identifying a specific nucleic acid sample. For instance 4 bp tags allow 4⁴=256 different tags. Using such an barcode, the origin of a PCR sample can be determined upon further processing or fragments can be related to a clone. Also clones in a pool can be distinguished from one another using these sequence based barcodes. Thus, barcodes can be sample specific, pool specific, clone specific, amplicon specific etc. In the case of combining processed products originating from different nucleic acid samples, the different nucleic acid samples are generally identified using different barcodes. Barcodes preferably differ from each other by at least two base pairs and preferably do not contain two identical consecutive bases to prevent misreads. The barcode function can sometimes be combined with other functionalities such as adapters or primers and can be located at any convenient position. A barcode is often used as a fingerprint for labeling a DNA fragment and/or a library and for constructing a multiplex library. The library includes, but not limited to, genomic DNA library, cDNA library and ChIP library. Libraries, of which each is separately labeled with a distinct barcode, may be pooled together to form a multiplex barcoded library for performing sequencing simultaneously, in which each barcode is sequenced together with its flanking tags located in the same construct and thereby serves as a fingerprint for the DNA fragment and/or library labeled by it. A “barcode” is positioned in between two restriction enzyme (RE) recognition sequences. A barcode may be virtual, in which case the two RE recognition sites themselves become a barcode. Preferably, a barcode is made with a specific nucleotide sequence having 0 (i.e., a virtual sequence), 1, 2, 3, 4, 5, 6, or more base pairs in length. The length of a barcode may be increased along with the maximum sequencing length of a sequencer.

As used herein, “primers” refer to DNA strands which can prime the synthesis of DNA. DNA polymerase cannot synthesize DNA de novo without primers: it can only extend an existing DNA strand in a reaction in which the complementary strand is used as a template to direct the order of nucleotides to be assembled. The synthetic oligonucleotide molecules which are used in a polymerase chain reaction (PCR) as primers are referred to as “primers”.

As used herein, the term “DNA amplification” will be typically used to denote the in vitro synthesis of double-stranded DNA molecules using PCR. It is noted that other amplification methods exist and they may be used in the present invention without departing from the gist.

As used herein, “aligning” means the comparison of two or more nucleotide sequences based on the presence of short or long stretches of identical or similar nucleotides. Several methods for alignment of nucleotide sequences are known in the art, as will be further explained below.

“Alignment” refers to the positioning of multiple sequences in a tabular presentation to maximize the possibility for obtaining regions of sequence identity across the various sequences in the alignment, e.g. by introducing gaps. Several methods for alignment of nucleotide sequences are known in the art, as will be further explained below.

The term “contig” is used in connection with DNA sequence analysis, and refers to assembled contiguous stretches of DNA derived from two or more DNA fragments having contiguous nucleotide sequences. Thus, a contig is a set of overlapping DNA fragments that provides a partial contiguous sequence of a genome. A “scaffold” is defined as a series of contigs that are in the correct order, but are not connected in one continuous sequence, i.e. contain gaps. Contig maps also represent the structure of contiguous regions of a genome by specifying overlap relationships among a set of clones. For example, the term “contigs” encompasses a series of cloning vectors which are ordered in such a way as to have each sequence overlap that of its neighbors. The linked clones can then be grouped into contigs, either manually or, preferably, using appropriate computer programs such as FPC, PHRAP, CAP3 etc.

As used herein “dCas9” is a Cas9 Endonuclease Dead, also known as dead Cas9, and is a mutant form of Cas9 whose endonuclease activity is removed through point mutations in its endonuclease domains.

As used herein “labeling” or “Fluorescent labeling” is a process of incorporating a fluorescent tag to a molecule or in a system to visualize the fluorescent tag, also known as a label or probe. Labeling is facilitated by enzymes including direct labeling enzymes and or by DNA polymerases. Examples of labeling enzymes include, for example, S-Adenosyl-1-methionine (AdoMet or SAM)-dependent methyltransferases, Taq polymerase, Vent polymerase, Klenow polymerase etc. Fluorescent dyes are covalently bound to biomolecules such as nucleic acids or proteins so that they can be visualized by fluorescence imaging. Suitable fluorescently labeled nucleotides that can be incorporated in a DNA of interest include, without limitation, Alexa Fluor® 555-aha-dCTP, Alexa Fluor® 555-aha-dUTP, Alexa Fluor® 647-aha-dCTP, Alexa Fluor® 647-aha-dUTP, ChromaTide®; Alexa Fluor®; 488-5-dUTP, ChromaTide® Alexa Fluor® 546-14-dUTP, ChromaTide® Alexa Fluor® 568-5-dUTP, ChromaTide® Alexa Fluor® 594-5-dUTP, ChromaTide® Fluorescein-12-dUTP, ChromaTide® Texas Red®-12-dUTP, Fluorescein-aha-dUTP, DY-776-dNTP, DY-751-dNTP, ATTO 740-dNTP, ATTO 700-dNTP, ATTO 680-dNTP, ATTO 665-dNTP, ATTO 655-dNTP, OYSTER-656-dNTP, Cy5-dNTP, ATTO 647N-dNTP, ATTO 633-dNTP, ATTO Rho14-dNTP, ATTO 620-dNTP, DY-480XL-dNTP, ATTO 594-dNTP, ATTO Rho13-dNTP, ATTO 590-dNTP, ATTO Rho101-dNTP, Texas Red-dNTP, ATTO Thio12-dNTP, ATTO Rho12-dNTP, 6-ROX-dNTP, ATTO Rholl-dNTP, ATTO 565-dNTP, ATTO 550-dNTP. 5/6-TAMRA-dNTP, Cy3-dNTP, ATTO Rho6G-dNTP, DY-485XL-dNTP, ATTO 532-dNTP, 6-JOE-dNTP, ATTO 495-dNTP, BDP-FL-dNTP, ATTO 488-dNTP, 6-FAM-dNTP, 5-FAM-dNTP. ATTO 465-dNTP, ATTO 425-dNTP, ATTO 390-dNTP and MANT-dNTP. Suitable fluorescently labeled nucleotides also include dideoxynucleotides (ddNTPs). Each of the listed labels used with dNTPs is suitable for use with ddNTPs (e.g., ATTO 488-ddNTP) and is intended to refer to either a dNTP or ddNTP. Methods for nick-labeling are known in the art and are described herein. See, e.g., Rigby, P. W. J., et al. J. Mol. Biol. 113:237, which is incorporated herein by reference.

“Fragmentation” refers to a technique used to fragment DNA into smaller fragments. Fragmentation can be enzymatic, chemical or physical. Random fragmentation is a technique that provides fragments with a length that is independent of their sequence. Typically, shearing or nebulisation are techniques that provide random fragments of DNA. Typically, the intensity or time of the random fragmentation is determinative for the average length of the fragments. Following fragmentation, a size selection can be performed to select the desired size range of the fragments

“Physical mapping” describes techniques using molecular biology techniques such as hybridization analysis, PCR and sequencing to examine DNA molecules directly in order to construct maps showing the positions of sequence features.

“Genetic mapping” is based on the use of genetic techniques such as pedigree analysis to construct maps showing the positions of sequence features on a genome

The term “genome”, as used herein, relates to a material or mixture of materials, containing genetic material from an organism. The term “genomic DNA” as used herein refers to deoxyribonucleic acids that are obtained from an organism or which are derived from an RNA genome such as a viral genome. The terms “genome” and “genomic DNA” encompass genetic material that may have undergone amplification, purification, or fragmentation.

The term “reference genome”, as used herein, refers to a sample comprising genomic DNA to which a test sample may be compared. In certain cases, reference genome contains regions of known sequence information.

The term “double-stranded” as used herein refers to nucleic acids formed by hybridization of two single strands of nucleic acids containing complementary sequences. In most cases, genomic DNA are double-stranded.

As used herein, the term “single nucleotide polymorphism”, or “SNP” for short, refers to single nucleotide position in a genomic sequence for which two or more alternative alleles are present at appreciable frequency (e.g., at least 1%) in a population.

The term “chromosomal region” or “chromosomal segment”, as used herein, denotes a contiguous length of nucleotides in a genome of an organism. A chromosomal region may be in the range of 1000 nucleotides in length to an entire chromosome, e.g., 100 kb to 10 MB for example.

The terms “sequence alteration” or “sequence variation”, as used herein, refer to a difference in nucleic acid sequence between a test sample and a reference sample that may vary over a range of 1 to 10 bases, 10 to 100 bases, 100 to 100 kb, or 100 kb to 10 MB. Sequence alteration may include single nucleotide polymorphism and genetic mutations relative to wild-type. In certain embodiments, sequence alteration results from one or more parts of a chromosome being rearranged within a single chromosome or between chromosomes relative to a reference. In certain cases, a sequence alteration may reflect a difference, e.g. abnormality, in chromosome structure, such as an inversion, a deletion, an insertion or a translocation relative to a reference chromosome, for example.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, the term “endonuclease” refers to enzymes which cleave a phosphodiester bond within a polynucleotide chain (for example, enzymes which have an activity described as EC 3.1.21, EC 3.1.22, or EC 3.1.25, according to the IUBMB enzyme nomenclature).

“Site-specific endonucleases”, also known as “restriction endonucleases” or “restriction enzymes” recognize specific nucleotide sequences in double-stranded DNA. Generally, endonucleases cleave both DNA strands of a DNA duplex. Some sequence-specific endonucleases can be engineered and/or modified to comprise only a single active endonuclease domain which cleaves only one of the strands in a DNA duplex and are thus referred to herein as “nicking endonucleases” or “nicking restriction endonucleases”. Nicking endonuclease catalyzes the hydrolysis of a phosphodiester bond, resulting in either a 5′ or 3′ phosphomonoester. Examples of nicking restriction endonucleases, such as those available from New England Biolabs, include Nb. BbvCI, Nt. BbvCI, Nt.Bsml, Nt.BsmAI, Nt.BstNBI, Nb. BsrDI, Nb.BstI, Nt.BspQI, Nt. BpulOI and Nt. BpulOI. The cleavage site or “nick site” of the phosphodiester backbone may fall within or outside of the recognition sequence, such as immediately adjacent the recognition sequence, of the site-specific nicking endonuclease.

An “RNA-guided endonuclease” includes those of the CRISPR-Cas (clustered regularly interspaced short palindromic repeats-(CRISPR) associated) adaptive immune systems found in roughly 50% of bacteria and 90% of archaea, as described, e.g., in Jiang and Doudna, Curr Opin Struct Biol. (2015) February:30:100-111 and Wright et al., Cell (2016) 164(1-2):29-44. RNA-guided endonucleases, such as Cas9, comprise two endonuclease domains. The HNH domain cleaves the target DNA strand whereas the RuvC domain cleaves the non-target DNA strand as defined by a so called “crRNA” strand bound by the endonuclease. According to certain aspects of the invention, the crRNA strand is generally comprised within a single-guide RNA (sgRNA).

As used herein, “nickase” refers to an enzyme which comprises a single active endonuclease domain which cleaves a single strand of DNA within a DNA duplex. In some embodiments, the nickase may be a mutant or variant form of a restriction endonuclease or of an RNA-guided endonuclease. For example, the nickase generally comprises an inactive endonuclease domain which does not cleave DNA, such as D10A Cas9 nickase, H840A Cas9 nickase, and the nicking restriction endonucleases such as Nb. BbvCI, Nt. BbvCI, Nt.Bsml, Nt.BsmAI, Nt.BstNBI, Nb. BsrDI, Nb. BstI, Nt.BspQI, Nt. BpulOI and Nt. BpulOI.

As used herein, “single guide RNA” or “sgRNA” refers to a single chimeric RNA which comprises the functions of a CRISPR RNA (crRNA) and a trans-acting crRNA known as tracrRNA (trRNA). The DNA cleavage site(s) of an RNA-guided endonuclease are within targeted DNA sequences defined by a 20 nt sequence within the sgRNA and adjacent to a PAM sequence within the DNA, as described in Jinek et al., Science (2012) 337:816-821.

### Methods

### CRISPR-Cas9 Enabled Whole-Genome Mapping

The CRISPR-Cas9 enabled whole-genome mapping is a universal multi-color mapping strategy in nanochannels that combines sequence-motif labeling system with Cas9 mediated target-specific labeling of any 20-base sequences (20mers) to create custom labels and detect new features present in DNA. Without wishing to be limited by theory, CRISPR-Cas9 enabled whole-genome mapping works by, labeling sequence motifs with, for example, green fluorophores: labeling the 20mers present within the DNA with, for example, red fluorophores: staining the DNA backbone with a backbone stain: imaging and analyzing the location of signals from each fluorophore and the backbone stain to map the entire genome. Using this strategy, it is not only possible to detect the SVs but it is also possible to interrogate the features not accessible to motif-labeling, locate breakpoints and precisely estimate copy numbers of genomic repeats.

In one aspect, the invention is a method of mapping a whole genome, wherein the method comprises the steps of labeling at least one DNA with a first fluorophore by contacting the at least one DNA with a solution comprising the first fluorophore and a labeling enzyme: nicking the at least one DNA labeled with the first fluorophore by contacting it with a solution comprising a nickase and at least one single guide RNA (sgRNA) or at least one crisprRNA (crRNA): incorporating fluorescent nucleotide(s) at the nicked site(s) of the at least one DNA by contacting it with a solution comprising a DNA polymerase and a mix of nucleotides comprising at least one nucleotide tagged with the second fluorophore: staining the backbone of the at least one nicked-labeled DNA with a DNA backbone stain: imaging the stained DNA by sequentially exciting the first fluorophore, the second fluorophore, and the DNA backbone stain; and analyzing the imaging data for identifying the location of the first fluorophore and the second fluorophore for genome mapping.

In certain embodiments, the at least one DNA is a genomic DNA (gDNA).

In certain embodiments, the enzyme is Direct Label Enzyme (DLE-1, Bionano Genomics).

In certain embodiments, the polymerase is, for example, taq DNA polymerase.

In certain embodiments, the first fluorophore is green fluorophore. In certain embodiments, the first fluorophore is a DL-green fluorophore (Bionano Genomics). In certain embodiments, the green fluorophore labels CTTAAG motifs of the at least one DNA.

In certain embodiments, the second fluorophore is a red fluorophore.

In certain embodiments, the mix of nucleotides comprises Atto647 dUTP, Atto647 dATP dGTP, dCTP.

In certain embodiments, the backbone stain is YOYO-1 stain.

In certain embodiments, the DNA is loaded on a chip for imaging on nanochannels.

In certain embodiments, the first fluorophore is exited prior to exiting the second fluorophore.

In certain embodiments, the second fluorophore is exited prior to exiting the first fluorophore.

In certain embodiments, red and green fluorophores are sequentially excited with 637 and 532 nm lasers, respectively, and then, the YOYO-1-stained DNA backbone is excited with a 473 nm laser. The imaging data is further analyzed for whole genome mapping.

In certain embodiments, the at least one sgRNA or crRNA comprises about 20 nucleotides long recognition sequence. In certain embodiments, the nickase is a Cas9 nickase including, for example, D10A or H840A nickase.

In certain embodiments, the method is useful for applications including detecting breakpoints, characterizing repetitive sequence, investigating mutagenesis, and quantifying copy numbers.

In certain embodiments, the method is used in quantifying D4Z4 copy number variations in, for example, 4q35 and 10q26 chromosome arms as well as in telomeres. In certain embodiments, the method allows mapping of haplotypes. For example, the method allows not only to distinguish the 4q35 and 10q26 regions of D4Z4, but also separate the two haplotypes of 4qA, and 4qB based on DLE signature.

In certain embodiments, the method is used for telomere labeling and length estimation.

In certain embodiments, the method allows detecting long interspersed elements with DLE-Cas9 multicolor mapping.

In certain embodiments, the method allows using multiple gRNAs to label multiple targets in a single assay.

In certain embodiments the genome is a prokaryotic genome. In certain embodiments, the genome is an eukaryotic genome.

In certain embodiments, the genome is a mammalian genome. In certain embodiments, the genome is a human genome.

### CRISPR-Cas9 Enabled Whole-Genome Sequencing

### Nick-Labeling

The invention further provides various methods of CRISPR-Cas9 enabled whole-genome sequencing. Without wishing to be limited by theory, the method works by assembling DNA molecules on micropatterned substrate in a microfluidic device: introducing one or more CRISPR-Cas9 nickase (Cas9 D10A or Cas9 H840A)/gRNA complexes to nick the DNA molecules at the 20 base recognition sites: incorporating fluorescent nucleotides at the nicking sites: imaging the labeled DNA and analyzing the imaging results. The steps of nicking, tagging, imaging, and analyzing are optionally repeated, each time with a newer set of CRISPR-Cas9/gRNA complexes.

Thus, in one aspect, the invention provides a method of sequencing whole genome, wherein in certain embodiments at least one DNA molecule is linearized on a micropatterned surface. In certain embodiments, a thin gel film is laid on top of the at least one DNA molecule. In certain embodiments, the micropatterned surface is then assembled in a microfluidic device. In certain embodiments, in cycle one, one or more, and for example, four different CRISPR-Cas9 nickase (Cas9 D10A or Cas9 H840A)/gRNA complexes are introduced to nick the at least one DNA molecule at the 20 base recognition sites. In certain embodiments, a polymerase is employed to incorporate the fluorescent nucleotides at the nicking sites and lastly the labeled molecules are imaged and analyzed. In certain embodiments, after imaging, the enzyme and gRNA are removed by protease and RNAase. In certain embodiments, the system can run many cycles and read the whole genome. In certain embodiments, the gRNAs are designed such that a different colored fluorescent nucleotide can be incorporated for each of the gRNAs.

Labeling without Nicking

In this method, instead of Cas9, dCas9 is used for forming fluorophore tagged gRNA/Cas9 complexes. Such dCas9/gRNA complexes bind to DNA recognition sites without nicking or cutting. After dCas9/gRNA complexes bind to recognition sites, imaging and analysis is performed. The labeling relies on the binding of fluorescent dCas9/gRNA complex to the specific DNA loci.

Thus, in another aspect, the invention provides a method of sequencing whole genome, wherein the method comprises steps of linearizing at least one DNA on a micropatterned surface: labeling the at least one DNA by contacting it with at least one dCas9/gRNA complex, wherein either the dCas9 or the gRNA is tagged with a fluorophore; and imaging and analyzing the labeled DNA. In certain embodiments, the tracrRNA is linked with a fluorophore. In certain embodiments, the dCas9 can bind to recognition sites without nicking or cutting.

In certain embodiments, different colored fluorophores are used for tagging dCas9/gRNA complex(es) comprising different gRNAs.

In certain embodiments the genome is a prokaryotic genome. In certain embodiments, the genome is an eukaryotic genome.

In certain embodiments, the genome is a mammalian genome. In certain embodiments, the genome is a human genome.

### Labeling Using Fluorophore-Tagged Reversible Terminators

In this method, the Cas9/gRNA complexes are used to create sequencing initiation sites (3′-OH ends) along DNA molecules that are linearized on a micropatterned surface: fluorophore-tagged reversible terminators are introduced to read single bases one incorporation at a time. Following the first incorporation, the 3′ modification is reversed to —OH to resume the second base addition. In this manner, base-by-base sequencing at the multiple initiation sites is performed along a single DNA molecule.

Thus, in yet another aspect, the invention provides method of sequencing whole genome, wherein the method comprises linearizing at least one DNA on a micropatterned surface: generating sequencing initiation site(s) (3′-OH ends) along the at least one DNA by contacting it with a solution comprising at least one Cas9/gRNA complex: labeling the at least one DNA by contacting it with a solution comprising a DNA polymerase and a mix of fluorophore-tagged reversible terminators: imaging the at least one DNA: reversing the 3′ modification to —OH. Repeating steps of reversing 3′ modification to —OH, labeling, and imaging the at least one DNA for sequencing the whole genome.

In certain embodiments, the Cas9 nickase includes, for example, D10A or H840A nickases.

In certain embodiments, each gRNA is designed to target hundreds of thousands of 20 base recognition sequences across the genome.

In certain embodiments, the at least one DNA is a megabase-long DNA. In certain embodiments, each reversible terminator comprising different nucleotides are tagged with different fluorophores.

Using the methods detailed above multiple molecules can be sequenced simultaneously in a single device

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

The materials and methods employed in the experiments disclosed herein are now described.

### Materials and Methods

### DNA Preparation

High molecular weight gDNA was purified either from cells embedded into agarose-gel plugs using commercial kits as per the manufacturer's specifications (BioRad no. 170-3592) or via nanobind disk-based solid phase extraction (Bionano Genomics). The DNA samples were then quantified on Qubit using AccuGreen™ Broad Range dsDNA Quantitation Kit (Biotium). DNA samples whose concentrations were in the range of 36-150 ng/uL were used for labeling.

### Guide RNA Sequences.

Telomere, 4qD4z4, 10qD4z4 probes were ordered from Integrated DNA Technology (IDT) as crRNA. The LINE-1 single guide RNA (sgRNA) mix was synthesized in the lab.

They are designed to target 20 bases starting at 97,1425,3660 and 5841 respectively for sgRNA_1 to sgRNA_4 in a full-length LINE-1 reference (Genbank L1.3: GenBank: L19088). For LINE-1 insertion detection, the experiment using LINE-1 and telomere guide RNAs were performed. The same experiment also provided the data for our telomere analysis reported in here. For D4Z4 characterization, the experiment using three guide RNAs (4q D4Z4, 10q D4Z4 and telomere) were performed. Here, the telomere guide RNA was included as a control for second-labeling step, but not analyzed. In another experiment, all gRNAs listed in the Table 1 were combined, it generated similar results.

TABLE 1
Targets used in DLE-Cas9 labeling of NA12878.

| Guide RNAs | 20-base recognition sequences |
|---|---|
| LINE-1 sgRNA 1 | GGTACCGGGTTCATCTCACT |
| | (SEQ ID NO: 1) |
| LINE-1 sgRNA 2 | CAAGTTGGAAAACACTCTGC |
| | (SEQ ID NO: 2) |
| LINE-1 sgRNA 3 | GCTTATCCACCATGATCAAG |
| | (SEQ ID NO: 3) |
| LINE-1 sgRNA 4 | GAAGGGGAATATCACACTCT |
| | (SEQ ID NO: 4) |
| Telomere | TTAGGGTTAGGGTTAGGGTT |
| | (SEQ ID NO: 5) |
| 4qD4Z4 | TGGGAGAGCGCCCCGTCCGG |
| | (SEQ ID NO: 6) |
| 10qD4Z4 | GAGAGCGAAGGCACCGTGCC |
| | (SEQ ID NO: 7) |

### Single Guide RNA Synthesis.

Four LINE-1 specific targets (Table 1) were encoded on a 55 base DNA oligo along with T7 promoter (5′-TTCTAATACGACTCACTATAG-3′ (SEQ ID NO: 8)) and overlap sequences (5′-GTTTTAGAGCTAGA-3′(SEQ ID NO: 9)) and ordered from IDT. An 80-base complementary oligo designed to hybridize to the overlap sequence was also ordered from IDT (5′-AAAAGCACCGACTCGGTGCCACTTTTTCAAGTTGATAACGGACTAGCCTT ATTTTAACTTGCTATTTCTAGCTCTAAAAC-3′ (SEQ ID NO: 10)). A 10 μM equimolar pool of 4 oligos was first made and mixed 10 μM of complementary oligo in presence of 1× NEBuffer 2.0 (New England Biolabs, NEB) and 2 mM dNTPs. The mix was incubated at 90° C. for 15 s followed by 43° C. for 5 min to promote hybridization. Double-stranded DNA was synthesized later by adding 5 U of Klenow exo (NEB) to the mix and incubating at 37° C. for 1 hr. Any remnant single-stranded DNA was then degraded by the addition of 10 U Exonuclease I (NEB) in 1×Exonuclease buffer and incubating at 37° C. for 1 hr. The synthesized dsDNA was purified using QIAquick Nucleotide Removal Kit (Qiagen) and quantified via absorbance spectroscopy and used for RNA synthesis subsequent use in a transcription reaction. The sgRNA mix of 4 LINE-1 targets was synthesized following the manufacturer's instructions in NEB HiScribe™ T7 High Yield RNA Synthesis Kit and using the above dsDNA. After transcription and DNAseI (NEB) treatment, the sgRNA was purified using spin columns (Monarch® RNA Cleanup Kit T2030, NEB) and quantified via absorbance spectroscopy before use in the labeling reactions.

### Dle-Cas9 Labeling.

First, about 750 ng of genomic DNA was labeled with DLS labeling kit (Bionano Genomics) as per the manufacturer's recommendations. In the second step, 300 ng of DLE-1 labeled DNA was nicked with Cas9D10A and subsequently labeled with Taq DNA polymerase. The crRNA and/or sgRNA used for the Cas9 mediated nicking reactions are listed in Table 1.

Briefly, a direct labeling enzyme master mix was prepared with Bionano Genomics' DLE kit components (Direct Labeling enzyme, 1×DLE reaction buffer, and DL-Green labeling mix) and added to DNA. The reaction was mixed well and incubated at 37° C. for 2 hours. After this incubation, excess protein, fluorescent entities, and salt in the reaction volume was depleted by performing membrane dialysis for up to 2 hours at room temperature in dark. A 100 nm hydrophilic membrane (EMD Millipore, VCWP04700) was chosen for efficient diffusion. Following this, recovered DNA was once again quantified with Qubit before proceeding to the second step.

For the second step, 0.5 uL of 50 μM crRNA and 0.5 uL of 0.5 μM tracrRNA (IDT) were first mixed and incubated on ice for 30 minutes. This incubation was omitted when using synthesized guide RNA. Then, 200 ng Cas9D10A was added to the 25 pmol RNA and incubated in 1×NEB Buffer 3.1 for 15 minutes at 37° C. Later, 300 ng of DLE-1 labeled DNA was added to this mixture, and a nicking reaction was performed at 37° C. for 1 hour. Nicked DNA was then labeled in the presence of 67 nM of nucleotides (Atto647 dUTP, At-to647 dATP dGTP, dCTP) with 5 U Taq DNA polymerase for 1 hour at 72° C. in 1× Thermopol Buffer (NEB). The nick-labeled sample was treated with Proteinase-K (Qiagen) at 50° C. for 30 minutes and prepared for loading on nanochan-nels i.e., a staining mix (with flow buffer, DTT, and DNA stain in Bionano Genomics DLS kit) was prepared according to Bionano Prep Labeling NLRS Protocol-30024, Rev K (bionanogenomics.com), added to sample, and incubated overnight at room temperature to promote staining.

### Imaging on Bionano NanoChannels.

The labeled sample was loaded on the Bionano Saphyr G1.2 chip and imaged using a ‘dual labeled sample’ workflow. Red and Green labels are sequentially excited with 637 and 532 nm lasers, respectively, and then, the YOYO-1-stained DNA backbone is excited with a 473 nm laser. For each experiment, 480 Gb data was collected. The raw molecule images were converted into BNX files and saved on Bionano Access. The molecules were first de novo assembled based on the green channel (DLE-1) reference. Red labels were later identified based on the expected location on the genome and further analyzed.

### Two-Color Data Analysis.

Red label locations, identified with “1” in the “LabelChannel” column in the Cmap files in this assembly, were extracted. This information, however, is not listed in the Xmap files since the de novo assembly is performed based on the green-channel map. The locations for these labels relative to other green labels on the same molecule are found in the BNX file as well as the Cmap files. Shortlisted molecules for analysis containing the expected pattern of green and red labels were extracted from both these files. The raw molecules from the BNX file without stretch-match were used to generate histograms.

### Multiple Color Cas9-Cas9 Labeling

The DNA (300 ng) was first nicked with 200 ng Cas9 nickase (D10A or H840A). The nicked DNA was then labeled with 5 U of DNA Taq Polymerase (NEB), 100 nM ATTO532-dUTP dAGC and 1×NEBuffer 3.1 (NEB) at 72° C. for 60 minutes. The sample was treated with 0.3 U of SAP (USB Products) at 37° C. for 10 minutes and then 65° C. for 5 minutes. The gRNA (2.5 μM) was incubated with 200 ng of Cas9 D10A again, 1×NEBuffer 3 (NEB), and 1×BSA (NEB) at 37° C. for 15 minutes. The green-labeled sample was then added to the reaction and incubated at 37° C. for 1 hour. The Cas9D10A nicks were labeled with 2.5 U of Taq DNA Polymerase (NEB), ATTO647n red dATP, and 1×NEBuffer 3.1 (NEB) at 72° C. for 60 minutes. The nicks were repaired with 20KU of Taq DNA Ligase (NEB), 1 mM NAD+ (NEB), 100 nM dNTPs, and 1×NEBuffer 3.1 (NEB) at 37° C. for 30 minutes.

gRNA Selection (Quantify On-Off-Target Labeling Efficiency).

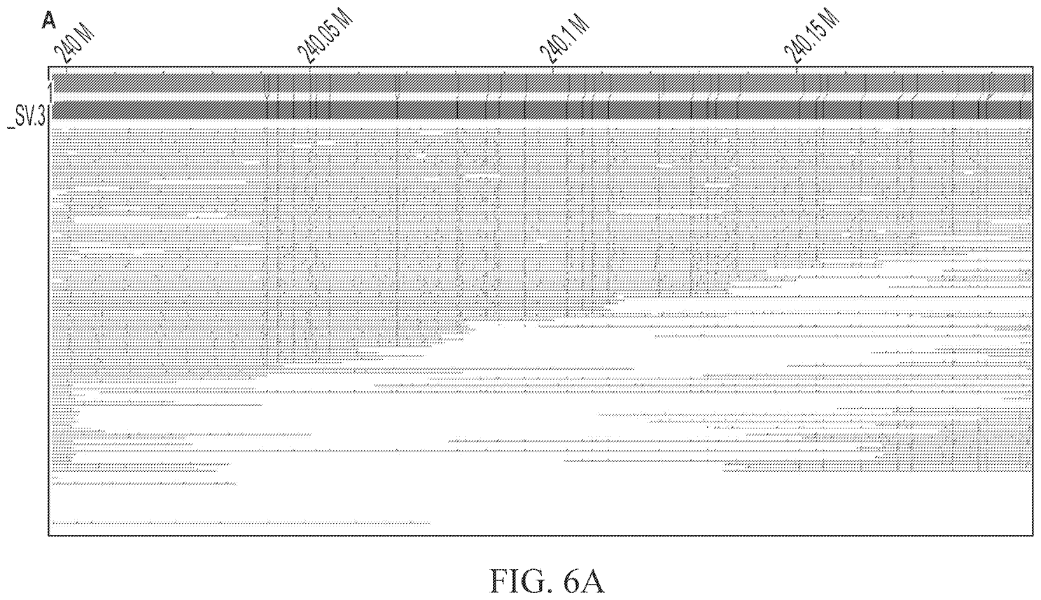

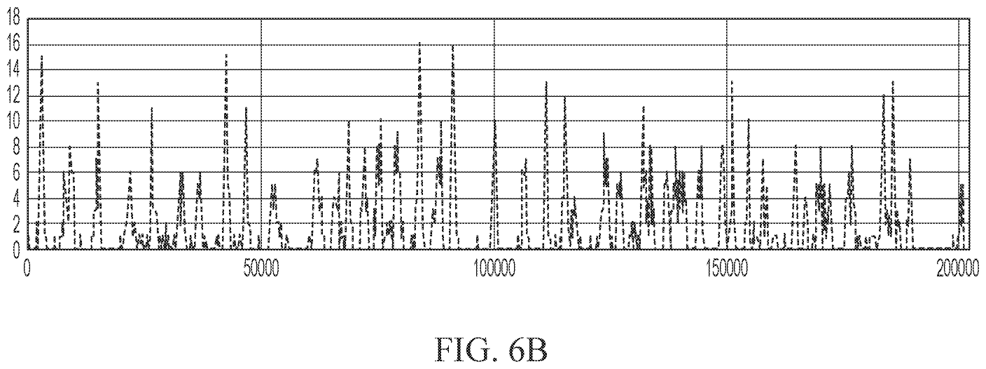

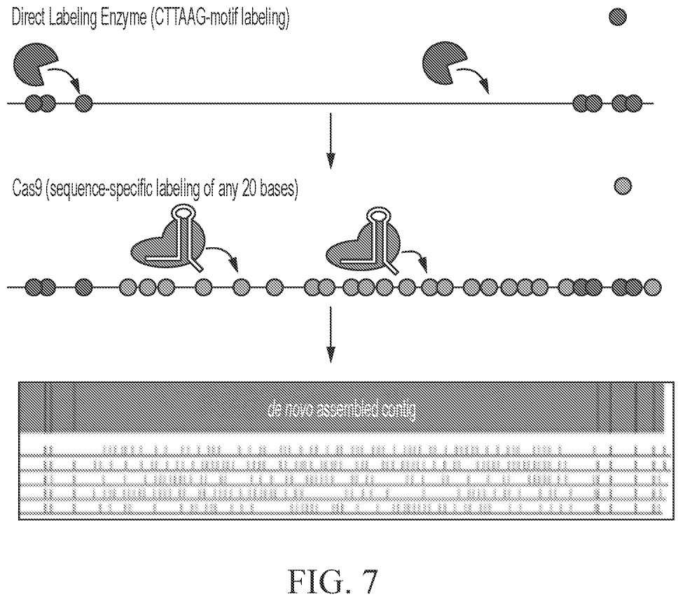

Multicolor labeling of DLE-Cas9 with many gRNAs was performed. Each experiment consists of one Cas9/gRNA and DLE labeling as shown in FIG. 6. The Cas9 labeling efficiency is defined as total red labels at a particular locus over the total number of molecules across the locus. 100% labeling means every molecule is labeled at that particular locus. A locus is labeled by Cas9 if the labeling efficiency is over 10% at a particular locus. The percentage of labeled loci is defined as the number of labeled loci over the total available loci. The results of four gRNAs are summarized in the Table 2 below. gRNAs can be selected based on the labeling efficiency and percentage of labeled loci. The gRNA4 is the best with the highest labeling efficiency and on-target labeling percentage. It also has the lowest off-target labeling percentage.

TABLE 2
quantifying on-off-target labeling efficiency

| | labeling | | | | | |
|---|---|---|---|---|---|---|
| | efficiency | Percentage of labeled loci | | | | |
| | On- | On- | Off- | Off- | Off- | |
| | target | target | target | target | target | |
| | No | No | 1 | 2 | 3 | |
| | muta- | muta- | muta- | muta- | muta- | |
| | tion | tion | tion | tion | tion | Total |
| Name of | in | in | in | in | in | loci |
| gRNA | 20 bp | 20 bp | 20 bp | 20 bp | 20 bp | labeled |
| gRNA1 | 45% | 89.63 | 36.96 | 33.01 | 20.29 | 525564 |
| (CGCCTGTAAT | | | | | | |
| CCCAGCACTT′ | | | | | | |
| (SEQ ID | | | | | | |
| NO: 11)) | | | | | | |
| gRNA2 | 33% | 97.68 | 44.34 | 18.56 | 5.86 | 214578 |
| (GCACTTTGGGA | | | | | | |
| GGCCAAGGC′ | | | | | | |
| (SEQ ID | | | | | | |
| NO: 12)) | | | | | | |
| gRNA3 | 84% | 98.16 | 69.67 | 52.68 | 3.26 | 166610 |
| (TTTCACCGTGT | | | | | | |
| TAGCCAGGA′ | | | | | | |
| (SEQ ID | | | | | | |
| NO: 13)) | | | | | | |
| gRNA4 | 90% | 98.48 | 44.27 | 14.56 | 2.21 | 399824 |
| (GCCTCAGCCTC | | | | | | |
| CCGAGTAGC′ | | | | | | |
| (SEQ ID | | | | | | |
| NO: 14)) | | | | | | |

### Example 1: Quantification of D4Z4 Copy Numbers in 4q35

The D4Z4 locus on the 4q35 chromosome arm is composed of tandemly repeating 3.3 kbp unit and D4Z4 copy number variation in 4qA is thought to be responsible for FSHD presentation. However, there is a high sequence homology (99.9%) of D4Z4 repeats among 10q26, and a 9.5 kbp region on Chr Y. This complicates the detection of copy numbers of D4Z4 repeats among these regions. Optical mapping relies on long single molecules of 300 kb, which is 10 times higher than the average read length of long-read sequencing methods.

In this experiment three guide RNAs (4q D4Z4, 10q D4Z4 and telomere) were used. The DNA was labeled at repeat motifs (CTTAAG) with green fluorophores using DLE enzyme. The D4Z4 repeat array was targeted using two guide RNAs-4qD4Z4 and 10qD4Z4 (Table 1). The telomere guide RNA as an internal control for second-labeling step. The two probes 4qD4Z4 and 10qD4Z4 (Table 1) were used to target the D4Z4 repeats on 4q chromosome arm with red fluorophores and are expected to generate a 1.68 kbp and 3.3 kbp repetitive label pattern. Based on the hg38 reference of 4q D4Z4 locus, the two target probes designed (‘4qD4Z4’ and ‘10qD4Z4’) generate the repeating units, the theoretical distance between is about 1648 bp. When one probe i.e., ‘4qD4Z4’ is used, a 3.3 kbp repeating unit will be detected and will result in the detection limit of one repeat unit. When two probes ‘4qD4Z4’ and ‘10qD4Z4’ are used, 1.68 kbp repeating unit is detected and the sensitivity will be half a repeat unit. This will increase the accuracy.

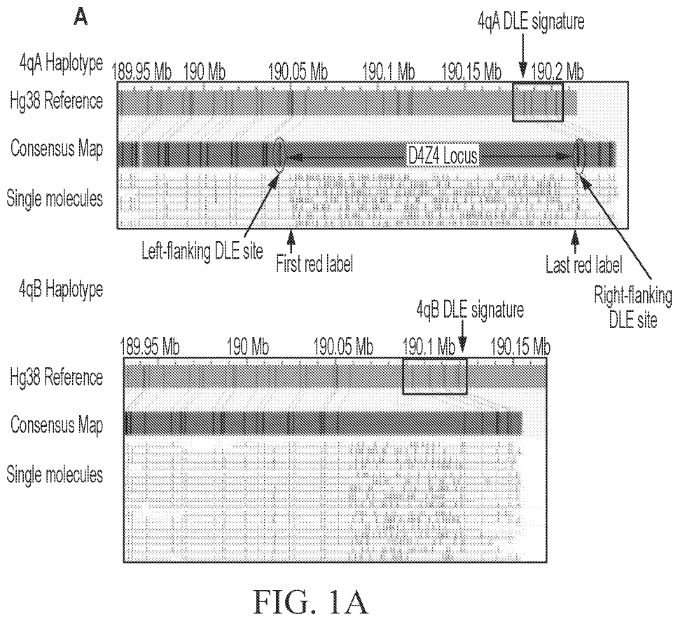

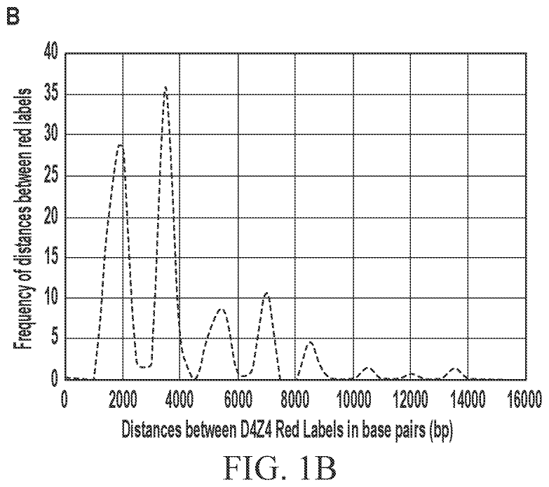

De novo assembled contigs spanning across D4Z4 regions are shown in FIG. 1A. DLE labels allow mapping not only to distinguish the 4q35 and 10q26 regions of D4Z4, but also separate the two haplotypes of 4qA, and 4qB based on DLE signature (FIG. 1A) (Bionano Solve Theory of Operation EnFocus FSHD Analysis Documentation, bi-onanogenomics.com). The molecules from 10q and 4q are already separated based on the DLE labels. The gRNAs were designed specifically to quantify the copy numbers of D4Z4 on the 4q chromosome.

The D4Z4 repeats labeling is shown as ticks in FIG. 1A. More red labels are present in the 4qA haplotype across longer distances than the 4qB haplotype. Varying distances between neighboring red labels are observed.

FIG. 1B shows the histogram of all recorded distances between neighboring red labels obtained from all molecules that span across the entire D4Z4 regions. The Gaussian fitting of each peak to find the peak locations at ˜1.68 kbp, 3.36 kbp, 5.0 kbp, 6.6 kbp. 9.9 kbp, and 13.2 kbp is then performed. A peak was observed at ˜1.68 kbp distance, shorter than the expected full D4Z4 repeat length, indicating that it was the distance between an on-target label and an off-target label. Longer distances, such as 6.6 kb, 9.9 kb, and 13.2 kb indicate that the expected red labels were missing. The average distance between all the peaks of halophyte 4qA, 1.68 kbp, was determined to be the average length of a D4Z4 repeating unit. Same 1.68 kb were obtained on the 4qB haplotype. This is exactly half of the 3.36 kb unit because of the off-target labeling due to the 10qD4Z4 probe. The red labeling at ˜190 Mb in FIG. 1A is probably due to the telomere-like sequence or off-target labeling of 4q D4Z4 guide RNA.

It was reasoned that the D4Z4 copy numbers can accurately be estimated by dividing the total length of D4Z4 from the first to last detected red labels by the 1.68 kb repeating unit. Using 1.68 kb as the repeating unit could increase the accuracy. To calculate the total length of D4Z4 repeats, it was needed to determine the ‘TRUE’ first and last red labels since the overall labeling efficiency within this array was not 100% and many molecules missed the first or last red label. The distances from the first red labels of each molecule to the left flanking DLE sites (arrows in FIG. 1A). 7.7 kb±2 kb is the shortest distance among 75% molecules belonging to the 4qA haplotype was measured. The same percentage of molecules on 4qA showed the distance between the last red label and the right flanking DLE sites to be 1 kb±2 kb. Only the molecules containing the TRUE″ first red label and TRUE″ last red label were used to calculate the total length of D4Z4 repeats. 37 molecules in 4qA and 44 molecules in 4qB, were used for our D4Z4 copy number analysis.

Taken all together, it was estimated that the 4qA has an average of 96 copies of 1.68 units and 48±0.94 copies of 3.36 kb units. The 4qB was estimated to have 38 copies of 1.68 units and 19±0.29 copies of 3.36 kb units. This is consistent with the numbers reported in previous studies. 30-32 Here, we showed the accuracy of less than a single copy.

FSHD is conventionally diagnosed using southern-blotting tests but they only offer semi-quantitative results. In a small set of the specimen (n=87), southern blotting tests produced indeterminate results in 23% of the cases. As a result, alternative molecular combing, optical mapping, and long-read sequencing-based approaches, for more efficient diagnosis of FSHD are gaining popularity. Although long-read sequencing read lengths have improved significantly since their inception, to date, whole-genome sequencing is expensive while targeted sequencing for long-regions, such as D4Z4 repeats remains infeasible. Optical mapping can address some issues with long molecules but, due to the lack of motifs within the array. D4Z4 repeats are estimated based on distances between closest DLE sites leading to inaccuracies. For more direct quantification, specific enzyme Nb. BssSI is needed, which tags each repeat with fluorophores. DLE-Cas9 is a more universal and versatile method, which can be used to tag any target or multiple targets simultaneously. The number of repeats that were estimated are comparable to earlier reports for healthy samples between 10-240. For the first time, the standard deviation of this method was quantified, 0.97 repeats for 4qA, which makes it possible to differentiate less than one D4Z4 repeat unit for 4qA (pathogenic haplotype). This is especially important for FSHD cases where the less than 8-10 repeats need to be counted accurately to differentiate the phenotypes.

### Example 2: Telomere Labeling and Length Estimation

Telomere length is a recognized clinical biomarker for aging and aging-related diseases. Several published studies correlate unregulated telomere length to malignant cancers (bladder, esophageal, gastric, head, breast, neck, ovarian, renal, and endometrial). The previously demonstrated optical mapping approach to estimate the individual telomere length by combining the conventional nickase-labeling with Cas9 labeling could map only 36 (out of 46) in the subtelomeric regions due to limitations like fragile sites (nick sites occurring close to each other on opposite strand). The two successive nicking reactions in the previous method are also laborious and cause DNA damage. To adequately address the above challenges, DLE-Cas9 methodology to perform a telomere length measurement assay is described herein.

In this assay, first Direct Label Enzyme (DLE-1, Bionano Genomics) was used to globally tag DNA at all DLE-specific motifs. For telomere-specific labeling, a Cas9 nick-labeling reaction was performed. The Cas9 nickase was directed to telomere repeats by a 20-base synthetic guide RNA ordered from IDT (Telomere, Table 1) to create nicks, and telomeric repeats were then labeled with red fluorescent dye. The labeled DNA molecules were imaged using high throughput nanochannel arrays on the Bionano Saphyr system. De novo assembly was performed based on the DLE-labels and the assemblies were aligned to hg38 reference. Individual molecules with red telomere labels at ends were identified and used for the quantification of telomere lengths.

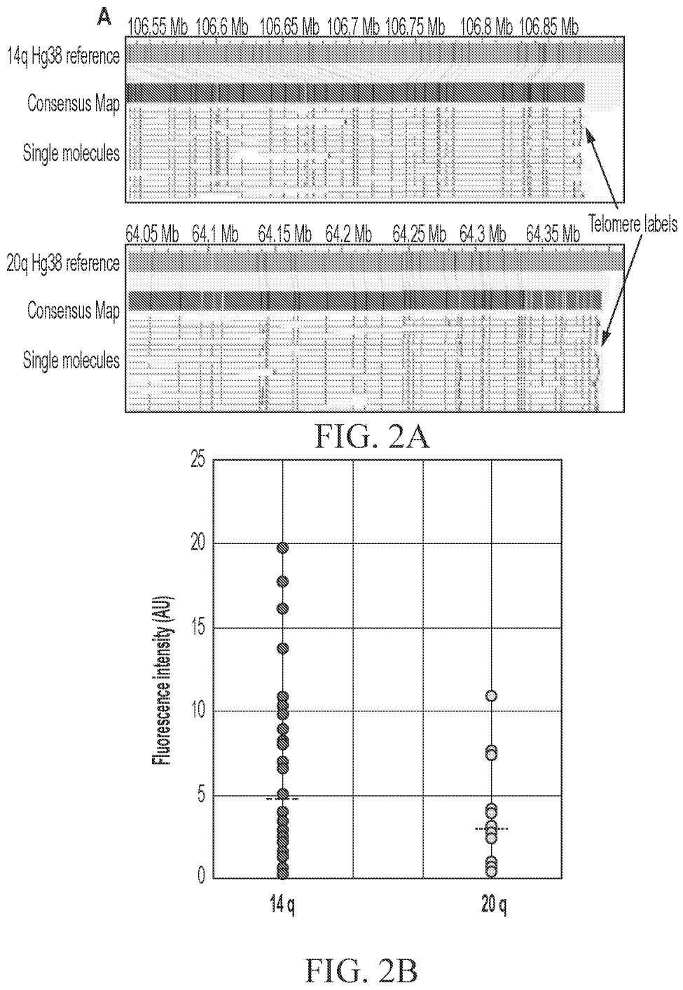

In FIG. 2A, the de novo assembled contigs of 14q and 20q with their long single molecules are shown aligned to hg38 reference. The wide bar at the top denotes the hg38 reference. The wide bar below the reference represents consensus contigs from the de novo assembly. The consensus contigs of both 14q and 20q matched well with the hg38 reference map. Individual molecules are represented by the thin lines arranged under the consensus contigs. Vertical ticks on the single molecules (thin lines) indicate labeled DLE sites and the other vertical ticks indicate target-specific red labels (shown by arrows). These red labels are clearly at the end of molecules indicating that the telomere repeats were labeled. In FIG. 2A bottom panel, the labeling at ˜64.27 Mb is due to the presence of telomere-like sequences in the subtelomeric region. As a proof of principle, the total intensity of telomere labels was then quantified from the molecules that belong to 14q and 20q arms, respectively. FIG. 2B shows a plot with measured intensities of red labels at telomere-termini containing single molecules. Each filled circle represents the total red label intensity of a single molecule. The 14q has an average intensity of 4.79±4.81, while 20q with an average intensity of 3.0±2.6. High standard deviations of intensity reflect the heterogeneity in telomere lengths from different cells within a sample. The fragmentation of either 5′ or 3′ telomere ends could affect the quantification. But they are a rare event among all telomere molecules and much less frequent than the DNA fragmentation in the middle, away from telomeres. Moreover, no telomere loss was observed (no telomere) normal cell lines as opposed to the telomere loss observed in cancer or aging cell lines. To translate the intensity to absolute base pairs, one needs to use a standard containing known telomere repeats and known system optical specificity. The lack of system information on the commercial system makes it difficult to provide basepair information.

Common telomere length assays include Terminal Restriction Fragment (TRF) and qPCR. Both methods estimate average telomere length. Single Telomere Length Analysis (STELA) and Quantitative fluorescence in situ hybridization (Q-FISH) were developed to detect and measure the length of specific telomeres. However, STELA can only measure a limited number of chromosomes and Q-FISH is limited in the analysis of cells currently in meta-phase and is unable to measure telomeres in terminally senescent cells or cells that are no longer able to divide.

Optical-mapping based telomere characterization assay can address the above challenges but due to fragile sites, has been successful in measuring only 36 of 46 telomere lengths. Using the assay described here in, it was possible to label and measure telomeric intensities in all chromosome arms except the 5 acrocentric chromosomes (data not shown). The lack of hg38 reference sequences makes it especially difficult to characterize the telomeres of the 5 remaining short acrocentric chromosome arms (13p, 14p, 15p, 21p, 22p). This methodology demonstrated the multiplex ability of targets in a single assay. All gRNAs listed in the Table 1 were combined to label multiple targets in a single assay, and it generated similar results (data not included). In an earlier report, the synthesis and use of up to 200 sgRNA in a single tube was demonstrated.

### Example 3. Detecting Long Interspersed Elements with DLE-Cas9 Multicolor Mapping

LINE-1 insertions make up ˜17% of the human genome. These insertions have been associated with various cancers, hemophilia, muscular dystrophy, and other genetic disorders. An individual is thought to have 80-100 active LINE-1 insertions responsible for most of the human retrotransposon activity. These active LINE-1s are ˜6 kbp in length and are thought to differ between individuals.

Optical mapping with sequence motifs, such as DLE, is very efficient in detecting insertions. When the size distribution of all insertions from the whole genome assembly is plotted, a peak at 6 kb is always observed, which could be mostly attributed to full-length LINE-1 insertions. However, optical mapping cannot differentiate other 6 kb insertions from LINE-1 insertions because mapping does not provide base-by-base information. As a proof of concept, DLE-Cas9 method is employed to tag and detect LINE-1 insertions in the NA12878 sample.

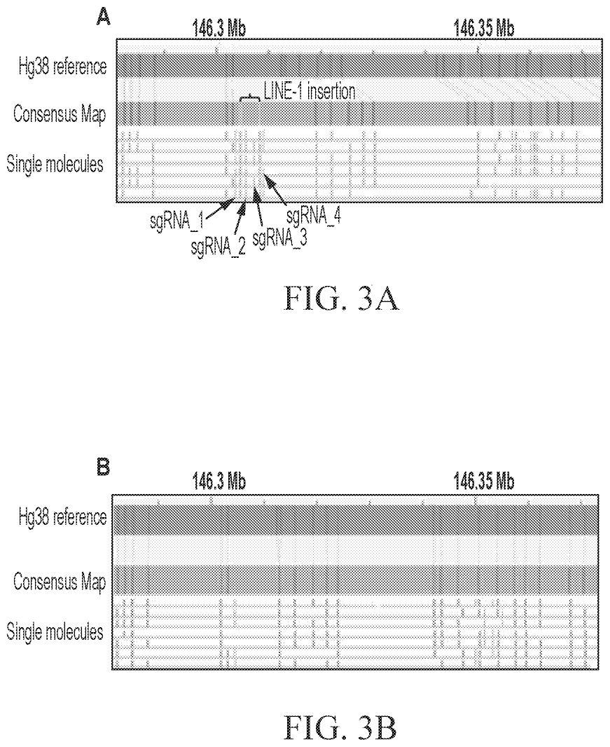

Single guide RNAs (Table 1) were designed and synthesized to target 4 different 20-base sequences on the LINE-1 reference at locations 97, 1425, 3660, 5841, and separated by 1328 bp, 2235 bp, and 2181 bp. These sites were labeled with red fluorescent nucleotides. De novo assembly was performed based on the DLE-labels and the assemblies were aligned to hg38 reference. A typical LINE-1 insertion detected using our DLE-Cas9 mapping is shown in FIG. 3. Here, both DLE and red labels have been stretch-matched and aligned to the reference.

Two haplotypes were observed in this region, with a 6 kb insertion detected from 146,303,137 bp to 146,312,443 bp in the haplotype 1 (FIG. 3A) with red labels and no insertion in haplotype 2 (FIG. 3B) at the same location. The average distances between red labels in haplotype were measured to be 1.5 kb, 2.3 kb, and 2.2 kb, which match the distances between the 4 designed guide RNA targets in a LINE-1 reference. The sequential 1.5-2.3-2.2 kb order also indicates the orientation of the insertion matches the reference. Moreover, the distances of two unmatched DLE motifs (yellow vertical lines on contig) inside the insertion also match the LINE-1 reference. Taken together, this insertion was designated as LINE-1 insertion. The other haplotype is shown without LINE-1 insertion (FIG. 3B) but may still have some LINE-1 like sequences because of the presence of some red labels. FIGS. 3A-3B also show some red labels in a neighboring location (from 146,347,677 bp to 146,357,405 bp), but without any detected insertion. These indicate the presence of some LINE-1 sequences in this location, near the LINE-1 insertion. Interestingly, many of the LINE-1 insertions occurred in the locations in the vicinity of LINE-1 sequences. The whole genome was then scanned to look for insertions with red labels that are separated by: 1.5 kb±0.5 kb, 2.3 kb±0.3 kb, and 2.3 kb±0.3 kb; only molecules with three red labels were used in the analysis. 55 LINE-1 insertion sites of NA12878 were discovered. These results were compared with a recent study by Zhou et al (Zhou, W. et al: Nucleic Acids Research 2019, 48 (3), 1146-1163) that identified LINE-1 insertions in NA12878 using PacBio sequencing data. The method presented herein was able to identify 51 of 52 of these insertions and 4 additional locations that were not reported by Zhou et al. On further investigation, it was discovered that the one location that was missed (chr2: 131243591-131243683) was not a true LINE-1 insertion since the optical maps did not show any insertions in this location nor were any red labels found. The four additional LINE-1 insertions all passed the pipeline. Table. 3 below lists all the locations with the zygosity and orientation where LINE-1 insertions were found. DNA molecules in nanochannels are typically stretched to 85% of their theoretical maximum length. However, factors like the width of the nano-channel salt concentration, voltage changes can cause localized variations in this stretching factor. However, a stretch-match function provided by Bionano Genomics was used to normalize the label locations in FIGS. 3A-3B. The stretch-match of red labels in FIGS. 3A-3B should not affect the LINE-1 detection. As four guide RNAs specific to LINE-1 sequences were used, the mere presence of the red labels together with the 6 kbp insertions detected by DLE labels should be enough to confirm that the insertions are LINE-1 sequences. In conclusion, sgRNA, labeling, and pipeline successfully detected all the LINE-1 insertions found by Zhou et al and found 4 new, previously unidentified locations.

Active LINE-1 insertions are frequent, non-static structural variations associated with cancer, neurologic and genetic disorders. Their mobile nature and variability between individuals make it challenging to study them. Long read sequencing, although is widely used to characterize LINE-1 insertions, produces low throughput and high cost may prevent its application in detecting specific LINE insertions. Sequence motif-based optical mapping, such as DLE and nickase do not provide sequence-level information for the identification of LINE-1 insertions. The applicability of DLE-Cas9 methodology for the detection and characterization of full-length LINE-1 insertions with their zygosity and orientation is demonstrated herein. This approach can benefit clinical investigations by providing haplotype-resolved and structurally accurate LINE-1 consensus maps for genomic analysis.

TABLE 3
LINE-1 insertions detected in NA12878 via the
DLE-Cas9 multi-color labeling methodology

| | | | | | |
|---|---|---|---|---|---|
| S. No. | Chr | Start | End | Orientation | Zygosity |

LINE-1 insertions detected by methods presented herein and by Zhou's method.

| | | | | | |
|---|---|---|---|---|---|
| 1 | 2 | 22964869 | 22970286 | − | Heterozygous |
| 2 | 2 | 35649838 | 35657550 | − | Heterozygous |
| 3 | 2 | 36339512 | 36350808 | − | Heterozygous |
| 4 | 2 | 81869209 | 81874699 | − | Heterozygous |
| 5 | 2 | 97155813 | 97160229 | − | Heterozygous |
| 6 | 2 | 155670566 | 155676303 | + | Heterozygous |
| 7 | 3 | 38582294 | 38592293 | + | Heterozygous |
| 8 | 3 | 55750771 | 55755088 | + | Homozygous |
| 9 | 3 | 85523459 | 85527546 | + | Heterozygous |
| 10 | 3 | 101557989 | 101567727 | + | Heterozygous |
| 11 | 3 | 123864357 | 123872447 | + | Homozygous |
| 12 | 3 | 143402794 | 143402963 | − | Heterozygous |
| 13 | 3 | 151418216 | 151431645 | − | Heterozygous |
| 14 | 3 | 186650273 | 186655454 | + | Heterozygous |
| 15 | 4 | 68700645 | 68712439 | − | Heterozygous |
| 16 | 4 | 131256005 | 131268849 | − | Heterozygous |
| 17 | 4 | 146303136 | 146312780 | + | Heterozygous |
| 18 | 5 | 21205332 | 21210673 | + | Homozygous |
| 19 | 5 | 33795549 | 33798136 | − | Heterozygous |
| 20 | 5 | 90146236 | 90160633 | + | Homozygous |
| 21 | 5 | 110141207 | 110146311 | − | Homozygous |
| 22 | 6 | 13500995 | 13504649 | + | Homozygous |
| 23 | 6 | 102396289 | 102401522 | − | Heterozygous |
| 24 | 6 | 123528514 | 123534095 | − | Heterozygous |
| 25 | 6 | 142128943 | 142129154 | − | Heterozygous |
| 26 | 6 | 157535053 | 157548815 | − | Homozygous |
| 27 | 7 | 7957100 | 7981363 | + | Heterozygous |
| 28 | 7 | 42487230 | 42491515 | − | Heterozygous |
| 29 | 7 | 53575730 | 53603976 | − | Heterozygous |
| 30 | 7 | 62333977 | 62334179 | − | Homozygous |
| 31 | 7 | 67117832 | 67145981 | − | Homozygous |
| 32 | 7 | 108184087 | 108189154 | + | Heterozygous |
| 33 | 9 | 91644707 | 91672990 | − | Heterozygous |
| 34 | 10 | 25418472 | 25418866 | − | Homozygous |
| 35 | 10 | 122694103 | 122696357 | + | Homozygous |
| 36 | 11 | 110497283 | 110510450 | − | Homozygous |
| 37 | 12 | 28065050 | 28078551 | − | Heterozygous |
| 38 | 12 | 117366349 | 117379186 | − | Heterozygous |
| 39 | 12 | 126318369 | 126318395 | − | Heterozygous |
| 40 | 13 | 60876288 | 60889129 | − | Homozygous |
| 41 | 13 | 106780129 | 106785630 | − | Heterozygous |
| 42 | 14 | 52194998 | 52200594 | − | Homozygous |
| 43 | 14 | 58749977 | 58754020 | + | Heterozygous |
| 44 | 15 | 33739015 | 33741207 | − | Heterozygous |
| 45 | 15 | 55958927 | 55959002 | + | Heterozygous |
| 46 | 17 | 66633343 | 66643120 | + | Heterozygous |
| 47 | 17 | 70355080 | 70366552 | + | Heterozygous |
| 48 | 18 | 15091008 | 15097533 | − | Homozygous |
| 49 | 21 | 8674532 | 8682071 | − | Heterozygous |
| 50 | X | 112307985 | 112318757 | + | Heterozygous |

| | | | | | |
|---|---|---|---|---|---|
| Index | Chr | Start | End | Orientation | Zygosity |

LINE-1 insertions uniquely detected by methods presented herein

| | | | | | |
|---|---|---|---|---|---|
| 51 | 2 | 143547387 | 143548599 | − | Heterozygous |
| 52 | 10 | 36467218 | 36479270 | + | Heterozygous |
| 53 | 12 | 33854180 | 33867084 | − | Homozygous |
| 54 | 18 | 12476887 | 12495587 | + | Heterozygous |

False negative detected by methods presented herein.

| | | | |
|---|---|---|---|
| 55 | 3 | 81941743 | 81941918 |

Deemed as not LINE-1 insertion by methods presented herein.

| | | | |
|---|---|---|---|
| 56 | 2 | 131243591 | 131243683 |

Legend for Table 3:
Columns ‘Chr’, ‘Start’ and ‘End’ list the chromosomes and locations where these insertions occur.
Column ‘Orientation’ identifies whether the LINE-1 insertion is inverted (−) or not (+).
Column ‘Zygosity’ refers to whether the LINE-1 insertion was found in only one contig/haplotype (Heterozygous) or both contigs/haplotypes (Homozygous) inthe given location.

### Example 4: Conclusions

The long-read sequencing technologies have been progressing tremendously since their inception. However, the lower throughput, high cost, high error rate, and still relatively short average read length still limited their application. For example, in estimating the D4Z4 repeat copy numbers, the read length must reach more than 300 kb including the upstream and downstream sequences to separate the different haplotypes. Optical mapping can read single molecules with an average length of 300 kb. Optical mapping also offers a cost advantage, one can obtain 200× coverage with about $500 comparing $10-20,000 for whole-genome sequencing with long-read technologies. targeted sequencing of D4Z4 is still challenging with no commercially available enrichment kit that can capture D4Z4. For the first time, the technological feasibility of combining DLE sequence-specific labeling and Cas9 mediated target-specific labeling to target any sequences in the genome is demonstrated herein. This is a universal and versatile methodology that can be used in the simultaneous analysis of multiple targets. In an earlier report, synthesis and use of up to 200 sgRNA in a single tube reaction was demonstrated: custom synthesizing the sgRNA significantly reduces the cost of assays. The method described herein can detect LINE-1 insertions, estimate the copy numbers of D4Z4 repeats and telomere length in a single tube reaction, with the combination of either crRNA or sgRNA. More importantly, the whole assay is built on the commercial instrument and assay kit.

### Example 5. CRISPR-Cas9 Enabled Whole-Genome Sequencing

### Method 1

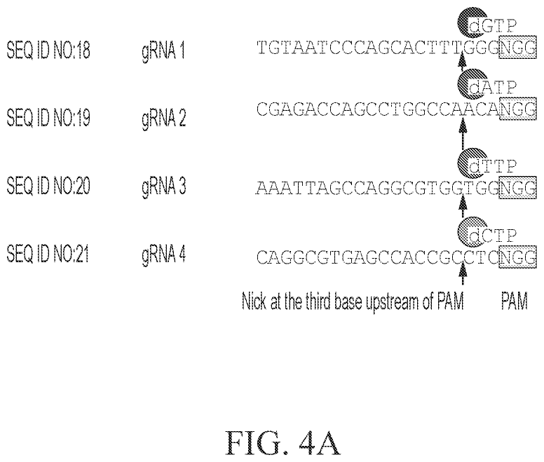

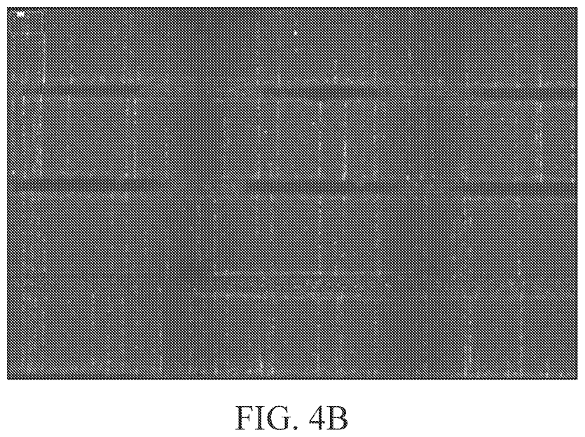

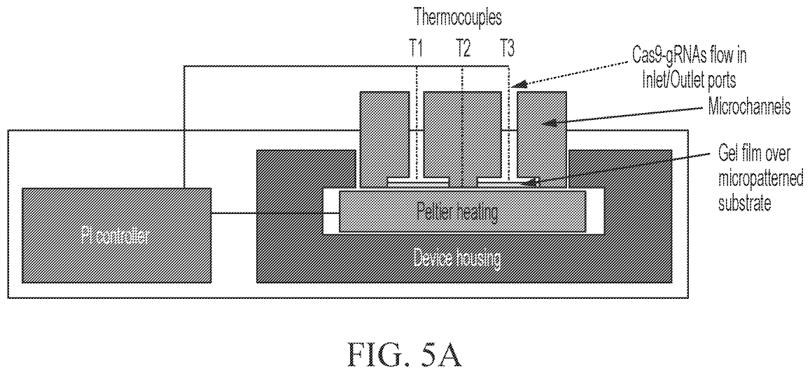

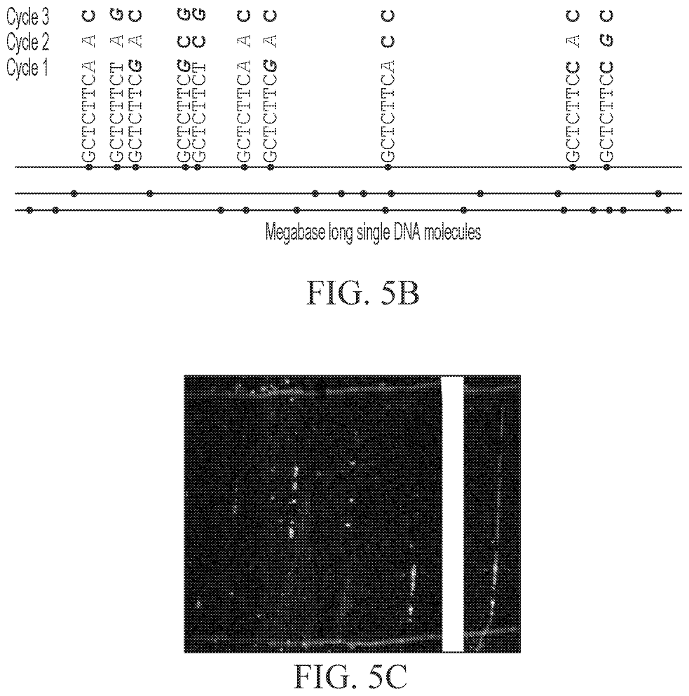

Long DNA molecules are linearized on a micropatterned surface, and a thin gel film is laid on top of the DNA molecules. The micropatterned surface is then assembled in a microfluidic device. In cycle one, one or more up to 4 CRISPR-Cas9 nickase (Cas9 D10A or Cas9 H840A)/gRNA complexes are introduced to nick the DNA molecules at the 20 base recognition sites. Then the polymerase will be employed to incorporate the fluorescent nucleotides at the nicking sites. The labeled molecules will be imaged and analyzed. Each gRNA is designed to target hundreds of thousands of 20 base recognition sequences across the genome. For example, the gRNA (CCCAGCACTTTGGGAGGCCG (SEQ ID NO: 15)) will have 500,000 sites containing the same sequence of CCCAGCACTTTGGGAGGCCG (SEQ ID NO: 16), while a different gRNA, (TTTCACCGTGTTAGCCAGGA (SEQ ID NO: 17)) targets over 100,000 loci. After imaging, the enzyme and gRNA will be removed by protease and RNAase. One or more up to 4 different CRISPR-Cas9 nickase/gRNA complexes will be introduced again to start cycle two. The system will be able to run many cycles and read the whole genome. FIGS. 4A-4B. shows a 4-color sequencing scheme combining 4 different gRNAs in a single cycle. The gRNAs are designed such that a different colored fluorescent nucleotide can be incorporated for each of the 4 gRNAs.

### Method 2

The procedure in this example is similar to the protocol in Example 4 except the Cas9 nickases are replaced by the dCas9, which can bind to the recognition sites without nicking or cutting. In the dCas9/gRNA complex, either the dcas9 is labeled with different color fluorophores or gRNAs are tagged with different color fluorophores.

### Method 3

In this example, the Cas9 (D10A or H840A)/gRNA complexes are used to create sequencing initiation sites (3′-OH ends) along a single megabase-long DNA molecule. To create these sites, the Cas9/gRNA complexes are flown into a microfluidic device where the megabase-long DNA molecules are linearized on a micropatterned surface. Next, after washing, a polymerase enzyme and fluorophore-tagged reversible terminators are introduced to read single bases, one incorporation at a time. Following the first incorporation, imaging was performed, and then reverse the 3′ modification to —OH to resume the second base addition. In this manner, base-by-base sequencing at the multiple initiation sites along a single DNA molecule was performed. There will be millions of such molecules being sequenced simultaneously in a single device.

### ENUMERATED EMBODIMENTS

The following exemplary embodiments are provided, the numbering of which is not to be construed as designating levels of importance:

Embodiment 1 provides a method of mapping a whole genome, wherein the method comprises:
a) labeling at least one DNA having a backbone with a first fluorophore by contacting the at least one DNA with a solution comprising the first fluorophore and a labeling enzyme;
b) nicking the at least one DNA labeled with the first fluorophore by contacting it with a solution comprising a nickase and at least one single guide RNA (sgRNA) or at least one crisprRNA(crRNA);
c) incorporating fluorescent nucleotide(s) at the nicked site(s) of the at least one DNA by contacting it with a solution comprising a DNA polymerase and a mix of nucleotides comprising at least one nucleotide tagged with the second fluorophore;
d) staining the backbone of the at least one nicked-labeled DNA of step c) with a DNA backbone stain;
e) imaging the at least one DNA of step d) by sequentially exciting the first fluorophore, the second fluorophore, and the DNA backbone stain; and
f) analyzing the imaging data to identify the location of the first fluorophore and the second fluorophore for whole genome mapping.

Embodiment 2 provides the method of embodiment 1, wherein the at least one DNA is a genomic DNA (gDNA).

Embodiment 3 provides the method of any embodiments 1-2, wherein the first fluorophore is a green fluorophore.

Embodiment 4 provides the method of any embodiments 1-3, where the first fluorophore labels CTTAAG motif(s) of the at least one gDNA.

Embodiment 5 provides the method of any embodiments 1-4, wherein the second fluorophore is a red fluorophore.

Embodiment 6 provides the method of any embodiments 1-5, wherein the first fluorophore is exited prior to exiting the second fluorophore.

Embodiment 7 provides the method of any embodiments 1-5, wherein the second fluorophore is excited prior to exciting the first fluorophore.

Embodiment 8 provides the method of any embodiments 1-7, wherein the at least one sgRNA or crRNA comprises an about 20 nucleotides long target-recognition sequence.

Embodiment 9 provides the method of any embodiments 1-8, wherein the nickase is Cas9D10A.

Embodiment 10 provides the method of any embodiments 1-9, wherein the backbone is stained with YOYO-1 stain.

Embodiment 11 provides the method of any embodiments 1-10, wherein the method is useful for applications including detecting breakpoints, characterizing repetitive sequence, investigating mutagenesis, and quantifying copy numbers.

Embodiment 12 provides a method of whole genome sequencing, the method comprises:
a) linearizing at least one DNA on a micropatterned surface;
b) nicking the at least one DNA by contacting it with a first solution comprising at least one CRISPR-Cas9 nickase/guide RNA (gRNA) complex;
c) incorporating fluorescent nucleotide(s) at the nicked site(s) of the at least one DNA of step b) by contacting it with a second solution comprising a DNA polymerase and a mix of nucleotides comprising at least one fluorescently tagged nucleotide;
d) imaging the at least one DNA of step c); and
e) repeating steps b)-d) with different CRISPR-Cas9 nickase/gRNA complex(es) than that used in previous steps for whole genome sequencing.

Embodiment 13 provides the method of embodiment 12, wherein the first solution comprises up to four different CRISPR-Cas9 nickase/gRNA complexes.

Embodiment 14 provides the method of any embodiment 12-13, wherein different colored fluorescent nucleotides are incorporated for different CRISPR-Cas9 nickase/gRNA complexes.

Embodiment 15 provides a method of whole genome sequencing, wherein the method comprises:
a) linearizing at least one DNA on a micropatterned surface;
b) labeling the at least one DNA by contacting it with a solution comprising at least one dCas9/gRNA complex tagged with a fluorophore; and
c) imaging and sequencing the labeled DNA.

Embodiment 16 provides the method of embodiment 15, wherein the dCas9 present in the dCas9/gRNA complex is tagged with a fluorophore.

Embodiment 17 provides the method of embodiment 15, wherein the gRNA present in the dCas9 nickase/gRNA complex is tagged with a fluorophore.

Embodiment 18 provides the method of any embodiments 15-17, wherein different colored fluorophores are used for tagging dCas9/gRNA complex(es) comprising different gRNAs.

Embodiment 19 provides a method of whole genome sequencing, wherein the method comprises:
a) linearizing at least one DNA on a micropatterned surface;
b) generating sequencing initiation site(s) (3′-OH ends) along the at least one DNA by contacting it with a first solution comprising at least one Cas9/gRNA complex;
c) labeling the at least one DNA from step b) by contacting it with a second solution comprising a DNA polymerase and a mix of fluorophore-tagged reversible terminators;
d) imaging the labeled DNA to read signal from the fluorophore;
e) reversing the 3′ modification to —OH;
f) repeating steps c)-e) and again step c); and
g) imaging the at least one DNA for whole genome sequencing.

Embodiment 20 provides the method of embodiment 19, wherein the at least one DNA is a megabase-long DNA.

Embodiment 21 provides the method of any of embodiments 19-20, wherein each reversible terminator comprising different nucleotides are tagged with different fluorophores.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of mapping a whole genome, wherein the method comprises:
a) labeling at least one DNA having a backbone with a first fluorophore by contacting the at least one DNA with a solution comprising the first fluorophore and a labeling enzyme;
b) nicking the at least one DNA labeled with the first fluorophore by contacting it with a solution comprising a nickase and at least one single guide RNA (sgRNA) or at least one crisprRNA(crRNA);
c) incorporating fluorescent nucleotide(s) at the nicked site(s) of the at least one DNA by contacting it with a solution comprising a DNA polymerase and a mix of nucleotides comprising at least one nucleotide tagged with the second fluorophore;
d) staining the backbone of the at least one nicked-labeled DNA of step c) with a DNA backbone stain;
e) imaging the at least one DNA of step d) by sequentially exciting the first fluorophore, the second fluorophore, and the DNA backbone stain; and
f) analyzing the imaging data to identify the location of the first fluorophore and the second fluorophore for whole genome mapping.

2. The method of claim 1, wherein the at least one DNA is a genomic DNA (gDNA).

3. The method of claim 1, wherein the first fluorophore is a green fluorophore.

4. The method of claim 2, where the first fluorophore labels CTTAAG motif(s) of the at least one gDNA.

5. The method of claim 1, wherein the second fluorophore is a red fluorophore.

6. The method of claim 1, wherein the first fluorophore is exited prior to exiting the second fluorophore.

7. The method of claim 1, wherein the second fluorophore is excited prior to exciting the first fluorophore.

8. The method of claim 1, wherein the at least one sgRNA or crRNA comprises an about 20 nucleotides long target-recognition sequence.

9. The method of claim 1, wherein the nickase is Cas9D10A.

10. The method of claim 1, wherein the backbone is stained with YOYO-1 stain.

11. The method of claim 1, wherein the method is useful for applications including detecting breakpoints, characterizing repetitive sequence, investigating mutagenesis, and quantifying copy numbers.

12. A method of whole genome sequencing, the method comprises:
a) linearizing at least one DNA on a micropatterned surface;
b) nicking the at least one DNA by contacting it with a first solution comprising at least one CRISPR-Cas9 nickase/guide RNA (gRNA) complex;
c) incorporating fluorescent nucleotide(s) at the nicked site(s) of the at least one DNA of step b) by contacting it with a second solution comprising a DNA polymerase and a mix of nucleotides comprising at least one fluorescently tagged nucleotide;
d) imaging the at least one DNA of step c); and
e) repeating steps b)-d) with different CRISPR-Cas9 nickase/gRNA complex(es) than that used in previous steps for whole genome sequencing.

13. The method of claim 12, wherein the first solution comprises up to four different CRISPR-Cas9 nickase/gRNA complexes.

14. The method of claim 12, wherein different colored fluorescent nucleotides are incorporated for each different CRISPR-Cas9 nickase/gRNA complexes.

15. A method of whole genome sequencing, wherein the method comprises:
a) linearizing at least one DNA on a micropatterned surface;
b) labeling the at least one DNA by contacting it with a solution comprising at least one dCas9/gRNA complex tagged with a fluorophore; and
c) imaging and sequencing the labeled DNA.

16. The method of claim 15, wherein the dCas9 present in the dCas9/gRNA complex is tagged with a fluorophore.

17. The method of claim 15, wherein the gRNA present in the dCas9 nickase/gRNA complex is tagged with a fluorophore.

18. The method of claim 15, wherein different colored fluorophores are used for tagging dCas9/gRNA complex(es) comprising different gRNAs.

19. A method of whole genome sequencing, wherein the method comprises:
a) linearizing at least one DNA on a micropatterned surface;
b) generating sequencing initiation site(s) (3′-OH ends) along the at least one DNA by contacting it with a first solution comprising at least one Cas9/gRNA complex;
c) labeling the at least one DNA from step b) by contacting it with a second solution comprising a DNA polymerase and a mix of fluorophore-tagged reversible terminators;
d) imaging the labeled DNA to read signal from the fluorophore;
e) reversing the 3′ modification to —OH;
f) repeating steps c)-e) and again step c); and
g) imaging the at least one DNA for whole genome sequencing.

20. The method of claim 19, wherein the at least one DNA is a megabase-long DNA.

21. The method of claim 19, wherein each reversible terminator comprising different nucleotides are tagged with different fluorophores.


SEQUENCE LISTING (21 sequences)

SEQ ID NO: 1 - DNA, 20 bp, Homo sapiens
ggtaccgggttcatctcact

SEQ ID NO: 2 - DNA, 20 bp, Homo sapiens
caagttggaaaacactctgc

SEQ ID NO: 3 - DNA, 20 bp, Homo sapiens
gcttatccaccatgatcaag

SEQ ID NO: 4 - DNA, 20 bp, Homo sapiens
gaaggggaatatcacactct

SEQ ID NO: 5 - DNA, 20 bp, Homo sapiens
ttagggttagggttagggtt

SEQ ID NO: 6 - DNA, 20 bp, Homo sapiens
tgggagagcgccccgtccgg

SEQ ID NO: 7 - DNA, 20 bp, Homo sapiens
gagagcgaaggcaccgtgcc

SEQ ID NO: 8 - DNA, 21 bp, Artificial Sequence
  T7 promoter
ttctaatacgactcactatag

SEQ ID NO: 9 - DNA, 14 bp, Artificial Sequence
  Overlap sequence
gttttagagctaga

SEQ ID NO: 10 - DNA, 80 bp, Artificial Sequence
  Oligo
aaaagcaccgactcggtgccactttttcaagttgataacggactagccttattttaacttgctatttctagctctaaaac

SEQ ID NO: 11 - DNA, 20 bp, Artificial Sequence
  Oligo
cgcctgtaatcccagcactt

SEQ ID NO: 12 - DNA, 20 bp, Artificial Sequence
  Oligo
gcactttgggaggccaaggc

SEQ ID NO: 13 - DNA, 20 bp, Artificial Sequence
  Oligo
tttcaccgtgttagccagga

SEQ ID NO: 14 - DNA, 20 bp, Artificial Sequence
  Oligo
gcctcagcctcccgagtagc

SEQ ID NO: 15 - DNA, 20 bp, Artificial Sequence
  Oligo
cccagcactttgggaggccg

SEQ ID NO: 16 - DNA, 20 bp, Homo sapiens
cccagcactttgggaggccg

SEQ ID NO: 17 - DNA, 20 bp, Artificial Sequence
  Oligo
tttcaccgtgttagccagga

SEQ ID NO: 18 - DNA, 20 bp, Artificial Sequence
  Oligo
tgtaatcccagcactttggg

SEQ ID NO: 19 - DNA, 20 bp, Artificial Sequence
  Oligo
cgagaccagcctggccaaca

SEQ ID NO: 20 - DNA, 20 bp, Artificial Sequence
  Oligo
aaattagccaggcgtggtgg

SEQ ID NO: 21 - DNA, 20 bp, Artificial Sequence
  Oligo
caggcgtgagccaccgcctc